(19) United States
(12) Patent Application Publication
(10) Pub. No.: US 2016/0029945 A1
(43) Pub. Date: Feb. 4, 2016

(54) VESTIBULAR TESTING

(71) Applicant: Massachusetts Eye & Ear Infirmary, Boston, MA (US)

(72) Inventors: Daniel Michael Merfeld, Lincoln, MA (US); Yongwoo Yi, Dorchester, MA (US)

(73) Assignee: Massachusetts Eye and Ear Infirmary, Boston, MA (US)

(21) Appl. No.: 14/775,421

(22) PCT Filed: Mar. 12, 2014
(86) PCT No.: PCT/US14/23988

§ 371 Date: Sep. 11, 2015

Related U.S. Application Data
(60) Provisional application No. 61/799,565, filed on Mar. 15, 2013.

(51) Int. Cl. A61B 5/00; A61B 5/16; A61B 3/10

(52) U.S. Cl. CPC A61B 5/4023; A61B 5/7275; A61B 3/10; A61B 5/164

(57) ABSTRACT

In one aspect, the disclosure features methods for estimating a vestibular function of a subject. The methods include moving the subject along a first direction parallel to the direction of gravity, receiving a first input set from the subject, the first input set indicating the subject's perception of the first direction, and estimating a first parameter related to a first vestibular function of the subject based on the first input. The methods further includes changing an orientation of the subject with respect to the earth, moving the subject along a second direction after changing the orientation of the subject, and receiving a second input set from the subject, the second input set indicating the subject's perception of the second direction.

### CLAIM OF PRIORITY

This application claims priority to U.S. Patent Application Ser. No. 61/799,565, filed on Mar.15, 2013, the entire contents of which are hereby incorporated by reference.

### STATEMENT OF GOVERNMENT RIGHTS

This work was supported in part by NIH/NIDCD grant DC04158, NIH grant R56DC012038, and NIH shared equipment grant 1S10RR028832. The United States government may have certain rights in the invention.

### BACKGROUND

The vestibular system of the inner ear enables one to perceive body position and movement. In an effort to assess the integrity of the vestibular system, it is often useful to test its performance. Such tests are often carried out at a vestibular clinic.

Vestibular clinics typically measure reflexive responses like balance or the vestibulo-ocular reflex (VOR) to diagnose a subject's vestibular system. The VOR is one in which the eyes rotate in an attempt to stabilize an image on the retina. Because the magnitude and direction of the eye rotation depend on the signal provided by the vestibular system, observations of eye rotation provide a basis for inferring the state of the vestibular system. Measurements of eye movement are useful for diagnosing some failures of the vestibular system. However, some patients report perceptual vestibular problems and still test normal on standard diagnostic tests that assess the VOR.

The failure of some VOR measurements might be because reflexive vestibular responses and vestibular perception use different neural pathways. Another reason may be that standard clinical measures focus on average VOR metrics like gain and phase. Other reasons may be that some disorders involve subtleties that are not assessed by measuring VOR. For example, VOR tests typically assess responses to motions with relatively large amplitudes, but it may also be important to conduct tests having motions with small amplitudes.

During a test at a vestibular clinic, some subjects feign poor performance for one reason or another. For example, a patient may feign test results to indicate that he or she has some form of disability to gain monetary advantages. As another example, a football player may feign test results when he or she is normal such that post-concussion test results can seem to be unchanged.

### SUMMARY

A subject (e.g., human or other animal) typically perceives direction of motion from visual and vestibular information. To assess a subject's ability to perceive motion, it is often useful to estimate a psychometric function (such as of vestibular function) that relates to the vestibular system of the subject. A collection of such psychometric functions can be used to generate a vestibulogram, which shows vestibular thresholds as a function of motion frequency.

The acquisition of data to estimate psychometric functions and to create a vestibulogram with sufficient accuracy can be time consuming The subject typically sits on a motion platform and presses buttons to signal his or her perception of motion. To generate a vestibulogram, data is collected across numerous motion frequencies and amplitudes—the subject endures this experience of almost complete sensory isolation for several hours.

This disclosure describes techniques and systems for estimating psychometric functions and using the estimated results to characterize the subject's ability to perceive motion. In some embodiments, a psychometric function is measured by translating the subject along a direction with a component parallel to gravity, during the vestibular test. The estimated psychometric function can provide a parameter (e.g., vestibular threshold) used to characterize the condition of the subject's ability to perceive motion.

In some embodiments, the techniques and systems disclosed herein enable monitoring the confidence rating of a received input from the subject, e.g., during the vestibular test. For example, the input device can enable the subject to input a binary response and a confidence rating of a perceived movement. The confidence rating can be correlated to the results of binary responses, where the correlation can improve the accuracy and/or decrease the overall time for conducting the vestibular test.

In one aspect, the disclosure features methods for estimating a vestibular function of a subject. The methods include, consist of, or consist essentially of: moving the subject along a first direction parallel to the direction of gravity, receiving a first input set from the subject, the first input set indicating the subject's perception of the first direction, and estimating a first parameter related to a first vestibular function of the subject based on the first input. The methods further include, consist of, or consist essentially of: changing an orientation of the subject with respect to the earth, moving the subject along a second direction after changing the orientation of the subject, and receiving a second input set from the subject, the second input set indicating the subject's perception of the second direction. Such methods include, consist of, or consist essentially of: estimating a second parameter related to a second vestibular function of the subject based on the second input and determining a relationship between the first parameter and the second parameter.

In some implementations, the first direction and the second direction can be substantially similar directions in a head coordinate of the subject. The second direction can be different from the direction of gravity. Determining the relationship can include comparing a magnitude of the first parameter and the second parameter.

In some implementations, the first direction and the second direction can be substantially different directions in a head coordinate of the subject. The second direction can be parallel to the direction of gravity.

In some practices, the methods can include evaluating whether the subject is a normal subject, a vestibular patient, or a malingerer based on the determined relationship between the first parameter and the second parameter.

Additional implementations can include producing a first vestibulogram based on the first parameter, producing a second vestibulogram based on the second parameter, and determining a relationship between the first vestibulogram and the second vestibulogram. Determining the relationship between the first vestibulogram and the second vestibulogram can include calculating a correlation function of the first vestibulogram and the second vestibulogram. The methods can include evaluating whether the subject is a normal subject, a vestibular patient, or a malingerer based on the correlation between the first vestibulogram and the second vestibulogram.

In another aspect, the disclosure features methods for estimating a vestibular function of a subject. The methods include, consist of, or consist essentially of: moving the subject along a direction parallel to the direction of gravity, receiving an input set from the subject, the input set indicating the subject's perception of the motion, estimating a first parameter related to a vestibular function of the subject based on the received input, determining a relationship between the estimated parameter and the predetermined parameter, and evaluating whether the subject is a normal subject, a vestibular patient, or a malingerer based on the relationship.

In some implementations, the subject can be evaluated to be a vestibular patient when the estimated parameter is significantly greater than the predetermined parameter or a malingerer when the estimated parameter is not significantly greater than the predetermined parameter.

In another aspect, the disclosure features methods for estimating a vestibular function of a subject. The methods include moving the subject along a direction, and receiving an input from the subject, the input indicating the subject's perception of the direction, where the input includes a reference response.

In another aspect, the disclosure features methods for estimating a vestibular function of a subject. The methods include, consist of, or consist essentially of: moving the subject along a direction, and receiving an input from the subject, the input indicating the subject's perception of the direction, where the input includes a binary response and a confidence rating.

In some implementations, the confidence rating can include any of a quasi-continuous rating, a binary rating, a N-level discrete rating, or a wagering rating. The methods can include fitting the confidence rating to a distribution function and determining a next motion of the subject based on the fit. The methods can include fitting the confidence rating to a distribution function to improve the estimation of the vestibular function.

In some practices, the methods can include determining a correlation between the binary response and the confidence rating, and evaluating the subject to be a malingerer if the determined correlation is different from a predetermined value.

In another aspect, the disclosure features methods for estimating a vestibular function of a subject. The methods include, consist of, or consist essentially of: moving the subject along a direction, and receiving an input from the subject, the input indicating the subject's perception of the direction, where the input includes a binary response. Such methods include, consist of, or consist essentially of: measuring vestibulo-ocular reflex (VOR) of the subject and producing VOR data from the measured VOR.

In some implementations, the methods can include fitting the VOR data to a distribution function and determining a next motion of the subject based on the fit. The methods can include fitting the VOR data to a distribution function to improve the estimation of the vestibular function. The methods can include determining a correlation between the binary response and the VOR data and evaluating the subject to be a malingerer if the determined correlation is different from a predetermined value.

In another aspect, the disclosure features apparatuses for estimating a vestibular function of a subject. The apparatuses include, consist of, or consist essentially of: a motion platform for supporting a subject, where the motion platform is configured to execute one or more motions, and an input device configured to receive a binary response and a reference response from the subject. The apparatuses further include, consist of, or consist essentially of: a processer configured to receive information from the input device based on the received binary response and reference response.

In some implementations, the reference response can include a confidence rating. The reference response can include a vestibulo-ocular reflex. The processer can be configured determine a relationship between the binary response and the reference response, where the relationship can be used to evaluate the motion sensing abilities of the subject. The relationship can be a correlation that is used to evaluate the motion sensing abilities of the subject.

In another aspect, the disclosure features apparatuses for estimating a vestibular function of a subject. The apparatuses include, consist of, or consist essentially of: a motion platform for supporting a subject, where the motion platform is configured to execute one or more motions, and an input device configured to receive confidence ratings from the subject. The apparatuses include, consist of, or consist essentially of: a processer configured to estimate a vestibular function by fitting a cumulative distribution to the confidence ratings.

In another aspect, the disclosure features apparatuses for estimating a vestibular function of a subject. The apparatuses include, consist of, or consist essentially of: a motion platform for supporting a subject, where the motion platform is configured to execute one or more motions, and an input device configured to measure the subject's VOR. The apparatuses include, consist of, or consist essentially of: a processer configured to fit a cumulative distribution to the VOR data.

In another aspect, the disclosed techniques include methods for estimating a psychometric function of a subject. The methods include, consist of, or consist essentially of: receiving an input from the subject, the input indicating the subject's response of a stimulus, where the input includes a binary response and a reference response.

The techniques and systems disclosed herein provide numerous benefits and advantages (some of which can be achieved only in some of the various aspect and implementations) including the following. Given the new systems and methods, information obtained by a translational motion along gravity can be used to improve results of vestibular tests for distinguishing between normal subjects (e.g., subjects without vestibular dysfunctions) and patients (e.g., subjects with vestibular dysfunctions). Estimated psychometric functions (as well as vestibulograms) obtained for directions along gravity, can be used to evaluate the motion sensing capabilities of a subject. For example, the subject can be evaluated to be either a normal subject, a patient with an actual disorder, or a malingerer. As used herein, a “malingerer” is a subject who does not have a substantial or significant (or any) psychometric disorders, but feigns a disorder or symptoms of a disorder by providing false responses to a test, typically for some monetary gain, e.g., insurance money or worker's compensation. In other words, malingerers try to “cheat” by feigning poor performance during a psychophysical test by intentionally indicating thresholds that are higher than normal.

In another aspect, the disclosed techniques include measuring binary responses as well as confidence ratings. In particular, the confidence ratings can be used to increase accuracy and testing time of psychometric tests. Correlation between confidence ratings and binary responses can also be used to evaluate whether a subject is normal, a patient, or a malingerer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages will be apparent from the following detailed description, and from the claims.

### BRIEF DESCRIPTION OF THE DRAWINGS

FIG. 1 is a schematic of a vestibular testing system.

FIGS. 2A-2C are schematics showing examples of body orientations along with corresponding head coordinates and earth coordinates.

FIGS. 3A and 3B are schematics showing examples of input devices.

FIG. 4A shows an example of an estimated psychometric function.

FIG. 4B shows additional examples of psychometric functions.

FIG. 5 is a flow chart depicting an example of a sequence of operations for estimating motion sensing ability of a subject.

FIG. 6 is a flow chart depicting another example of a sequence of operations for estimating motion sensing ability of a subject.

FIG. 7 is a flow chart depicting an example of a sequence of operations for receiving confidence ratings from a subject.

FIGS. 8A to 8D are a series of plots showing peak stimulus velocity at measured vestibular thresholds for normal subjects.

FIGS. 9A to 9D are a series of plots showing peak stimulus velocity at measured vestibular thresholds for subjects with vestibular dysfunctions.

FIGS. 10A and 10B are two plots showing peak stimulus velocity at measured vestibular thresholds for both normal subjects and subjects with vestibular dysfunctions.

FIG. 11 is a block diagram of a computing device.

FIGS. 12A-12C show example plots illustrating effects of providing distracting motions prior to providing motion stimuli.

### DETAILED DESCRIPTION

The methods and systems described herein can be implemented in many ways. Some useful implementations are described below. The scope of the present disclosure is not limited to the detailed implementations described in this section, but is described in broader terms in the claims.

Behavior of a subject can be assayed using psychophysical methods. For example, human behavior can be quantitatively represented using estimated fit parameters that characterize human psychophysical responses to a psychometric test. Quantitative assays that are robust, accurate, precise, and efficient, can be used for diagnostic purposes. Efficiency can be determined as, for example, a function of time or number of trials used in a psychometric test needed to yield a robust, accurate, and precise estimate of the fit parameter(s) that characterize psychophysical responses of the subject.

In particular, vestibular testing can be performed to estimate psychometric functions that represent the motion sensing abilities of the subject. Such testing can provide information related to a vestibular function of the subject. This specification discloses techniques for improving the accuracy and efficiency of estimating psychometric functions related to the vestibular system of the subject. The vestibular thresholds of normal subjects and subjects with vestibular dysfunctions can differ for a particular type of motion. Moreover, the threshold differences can be substantially larger for motions parallel to gravity than for motions that are not parallel to gravity. Therefore, estimating thresholds for motions parallel to gravity may provide more pronounced results for subjects with disorders. Moreover, by comparing thresholds measured for motions parallel with gravity and perpendicular to gravity, the disclosed techniques can be used to evaluate whether the subject has an actual disorder or is a likely to be a malingerer.

Vestibular testing can include VOR tests, which were described earlier. Such tests can provide information related to a vestibular function of a subject.

Accordingly, in some implementations vestibular tests can be used to measure vestibular functions of a subject. The vestibular functions can include information from a psychometric function and/or a vestibulometric function of the subject. The vestibular psychometric function can be obtained from psychometric tests evaluating the perceptive response of the subject. The vestibulometric functions can be obtained from, for example, VOR tests evaluating the reflex response of the subject.

Vestibular System

The vestibular system is the sensory system that provides the leading contribution to a subject's sense of movement and sense of balance. Being situated in the labyrinth in the inner ear of the subject, the vestibular system contributes to balance and to the sense of spatial orientation. The inner ear includes the vestibular labyrinth and the cochlea, which is the subject's hearing endorgan. For perceiving rotational and translational motions, the vestibular system includes two components: the semicircular canals, which sense rotational movements; and the otoliths, which sense linear accelerations and gravity. The vestibular system sends signals to the neural structures that control eye movements, and to the muscles that keep a creature upright. The signals sent for controlling eye movements form the anatomical basis of the VOR, which is required for clear vision. The signals sent to the muscles that control posture help keep the subject upright.

Example of a Vestibular Testing System

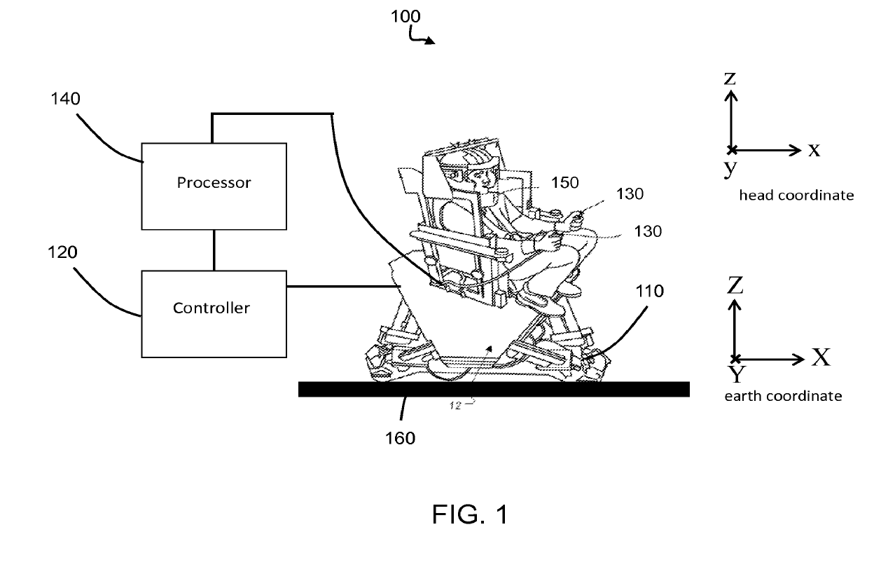

FIG. 1 shows an example of a vestibular testing system 100, which includes a motion platform 110 (e.g., a MOOG series 6DOF2000e), a controller 120 for controlling the motion of the motion platform 110, and an input device 130 for receiving input from a subject 150 whose vestibular system is to be tested. The processor 140 can receive input information from the input device 130 and may provide instructions to the controller 120 for moving the motion platform 110. During operation, the motion platform 110 supports the subject 150 and the controller 120 can provide a stimulus signal to the motion platform 110 for movement. Is some implementations, the processor 140 can be integrated with the input device 130.

Generally, each motion of the motion platform 110 can be described by a motion profile that includes information about the direction of motion and other features related to the motion. For example, a motion can be a translational motion along any of the three perpendicular axes x, y, and z of a coordinate system centered on head of the subject 150. Referring to FIG. 1, the x axis is pointing forward from the head, the y axis is pointing left from the head (into the drawing plane), and the z axis is pointing upward from the head. Such coordinate system respect to the head is referred as the “head coordinate” in this specification.

The motion profile can include amplitude and frequency of the velocity and acceleration of the motion. The amplitude of the acceleration and velocity vary with time, whereas the frequency remains constant. For example, a translational motion starts with a zero velocity, accelerates to a maximum velocity, and decelerates to zero again. For example, the acceleration is sinusoidal and can be expressed as

a(t)=A sin(2π ft) (1)

Where a(t) is the acceleration at time t, A is the acceleration amplitude, and f is the frequency. With such acceleration, starting from zero, the translational velocity v(t) at time t is

v(t)=A/(2π ft)[1−cos(2π ft)] (2)

Similarly, a rotational motion can include a sinusoidal angular acceleration and an angular velocity, both of which are expressed in a manner similar to the translation acceleration and velocity of Eqs. (1) and (2).

The motion platform 110 moves the subject along a trajectory in a spatial coordinate system while following a velocity profile. The velocity profile relates the magnitude of velocity to time. At the beginning and end of the motion, the magnitude of the velocity is zero. At some point in between, the velocity reaches a maximum magnitude, referred to herein as “peak velocity” or “peak stimulus velocity.” In many applications, the velocity profile is one cycle of such a velocity oscillation. The reciprocal of the period of this sine wave is referred to herein as “frequency” or “motion frequency.” As noted above, the shape of the velocity profile can be sinusoidal. However, other shapes are possible, such as those defined by superpositions of weighted and/or timeshifted components.

The motion platform 110 can have a translational motion in either x, y, or z direction. Accordingly, the translation motion in either direction is referred as “x-translation”, “y-translation”, or “z-translation”, respectively. In addition, the motion platform can have various rotational motions. Rotation about the x axis is referred as “roll” rotation, rotation about the y axis is referred as “pitch” rotation, and rotation about the z axis is referred as “yaw” rotation. The movements can be caused by the stimulus signal provided by the controller 120.

In some implementations, the controller 120 can change the orientation of the motion platform 110. Alternatively, a person can manually change the orientation. For example, the motion platform can be rotated 90 degrees to the side such that the subject 150 is lying on his or her side. Considering the variety of orientations of the motion platform 110, it is useful to refer a motion of the motion platform 110 (or the subject 150) using X, Y, and Z coordinates with respect to the fixed earth 160 (or ground.) Such coordinates are referred as “earth coordinates” in this specification. The Z direction is referred as “earth-vertical” and either the X or Y direction is referred as “earth-horizontal”.

In the example illustrated in FIG. 1, the X axis refers to a direction parallel to the ground, and the Y axis refers to another direction parallel to the ground, but perpendicular to the X axis. The Z axis points vertical to the ground. In this example, the head coordinates x, y, and z axes coincide with the earth coordinates X, Y, and Z axes. The illustrated body orientation of subject 150 is referred as the “upright position”.

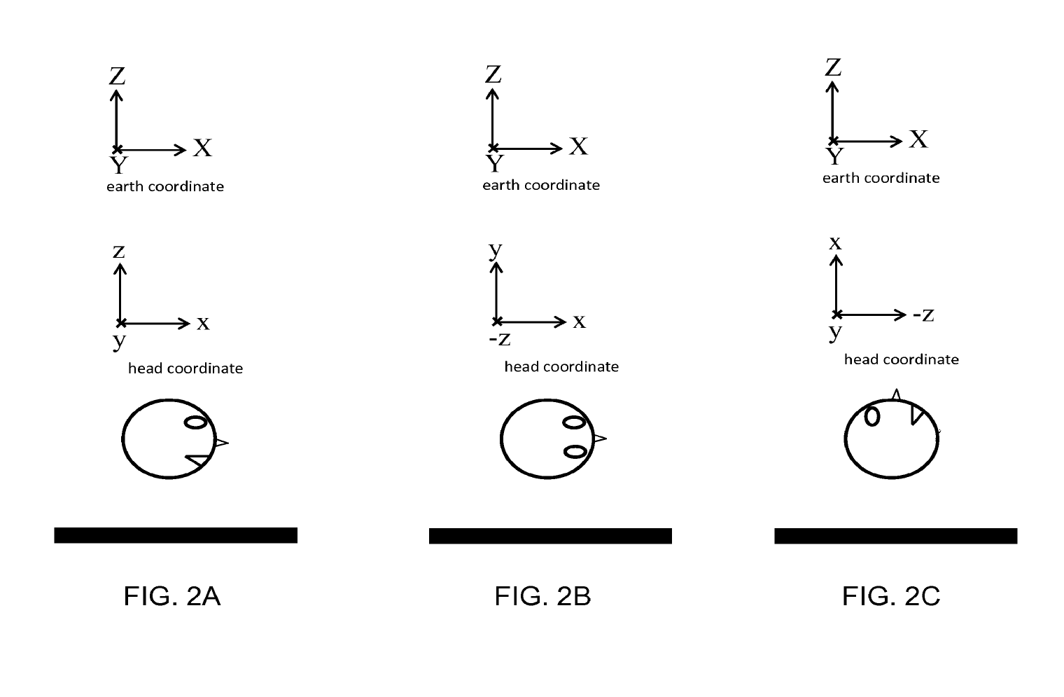

In some implementations, the motion platform 110 can be moved to be oriented such that the body orientation of the subject 150 is different from the upright position. FIGS. 2a-c shows a schematic of three different body orientations. FIG. 2A shows the up-right position previously described. FIG. 2B shows a “side-up position” where the motion platform 110 is rotated by 90 degrees such that the right side of the head is pointing towards the ground. In this orientation, the z axis may coincide with the −Y axis and the y axis may coincide with the Z axis. Alternatively, the left side of the head may point towards the ground. FIG. 2C shows a “back-down position” where the back of the head is pointing towards the ground. In this orientation, the x axis may coincide with the Z axis and the −z axis may coincide with the X axis. A “front-down position” refers when the front of the head is pointing towards the ground. Accordingly, the motion platform 110 may move the subject 150 in a variety of configurations depending on the body orientation, type, or direction of motion in head coordinates. In some implementations, the motion platform 110 can be configured to provide only one or several types of motions and body orientations. In this specification, a motion along, or aligned with, a specific direction may refer to motion in positive and negative directions of the specific direction. Similarly, a motion parallel to a specific direction may refer to motion which is parallel or antiparallel to the specific direction.

Examples of an Input Device in a Vestibular Testing System

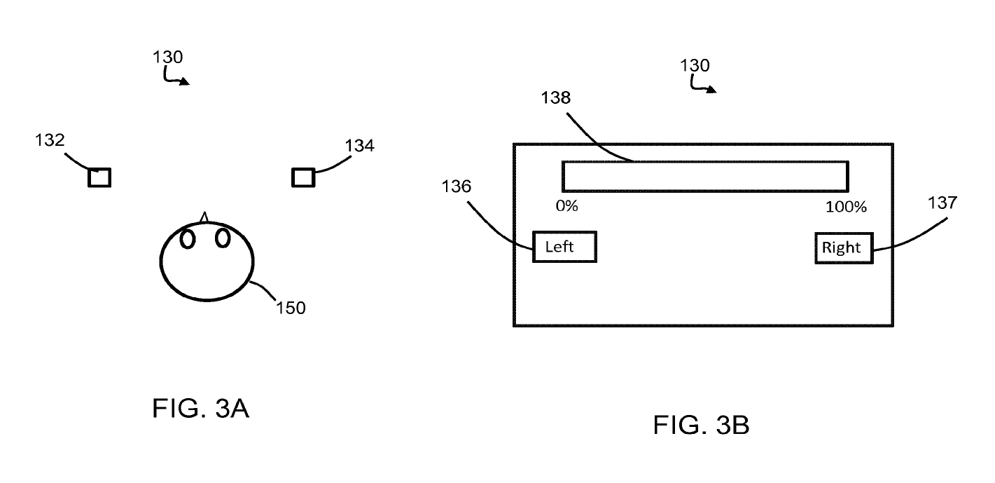

During operation, the subject 150 provides an input to the input device 130 to communicate his or her perception of motion to the processor 140. FIG. 3A shows an example of an input device 130, which includes a pair of buttons 132 and 134. Other examples of input device 130 include a joystick, pair of joysticks, a keyboard, a pair of switches, or foot pedals. After a motion of the motion platform 110, the subject 150 can press one of the buttons 132 and 134 to indicate his or her perception. For example, a particular button pressed can indicate the subject's perception of the motion's direction. In some examples, the subject 150 can press button 132 upon perceiving an upward translational motion and press button 134 when perceiving a downward translational motion.

FIG. 3B shows another example of an input device 130, which can be a touch screen such as a tablet device or a keyboard, e.g., a numeric keypad. The subject can indicate his or her perception by pressing either location 136 or 137 on the input device 130. For example, after a y-translation motion, the subject 150 can select location 136 if he or she perceives motion to his or her left. Alternatively, the subject 150 can select location 127 if he or she perceives motion to his or her right. As another example, after a z-translation motion, the locations 136 and 137 can be indicative of “up” or “down,” respectively. In some implementations, the input device 130 can simultaneously display more than two locations indicative of several types of motion (e.g., “left”, “right”, “up”, “down”, “translation”, “rotation”, etc.) In some implementations, the subject 150 can input his or her perception of a motion by swiping the display of the input device 130. For example, the subject 150 can swipe his or her fingers on the display to the left to indicate that the perceived motion is to his or her left direction.

In the example shown in FIG. 3B, the input device 130 includes a confidence rating menu 138. The subject 150 can indicate his or her confidence rating of the perceived motion using the confidence rating menu 138. In this example, the confidence rating menu is a quasi-continuous rating menu where 0% to 100% indicates the level of confidence in 1% increments. A quasi-continuous rating between 50% (guessing) and 100% (certain) is another example. Other ranges can be used. As described below, various types of confidence ratings other than the quasi-continuous rating can be used. In some implementations, the confidence rating menu 138 can be designed according to the type of confidence rating to be used.

In some implementations, the input device 130 can receive a binary response from the subject 150 through locations 136 and 137. After receiving the binary response, the input device 130 can further receive a confidence rating through the confidence rating menu 138. For example, the subject 150 can augment his or her binary response by providing a confidence rating including: (1) a quasi-continuous rating (e.g., 50% confidence to 100% confidence); (2) a binary rating (e.g., guessing versus certain); (3) a quinary rating (e.g., 1 to 5 where 1 is “guessing” and 5 is “certain,” or vice versa) or an N-level discrete rating (e.g., 1 to N where 1 is “guessing” and N is “certain” or vice versa); or (4) a wagering rating. The confidence rating can also be a combination of the forms (1)-(4). As described elsewhere herein, the received confidence rating can be used to: (1) improve the quality of estimating the psychometric function; (2) improve the efficiency of targeting stimulus levels in real-time via a closed-loop system during psychometric test; (3) reduce the negative impacts of indecision; (4) help evaluate subject's with psychometric (e.g., vestibular) dysfunctions; or (5) help evaluate malingerers. It is also understood that the confidence rating can be received before or simultaneous with the binary response.

As described above, the input device 130 can receive both the binary response and the confidence rating for a given motion, in other words, for each trial. The received data (e.g., binary response, confidence rating) can be communicated to the processor 140. The processor 140 can estimate a psychometric function and its threshold based on the communicated data. The communication can be done in a wired or wireless (e.g., WiFi, Bluetooth, or Near Field Communication) manner

The controller 120 can instruct (e.g., by providing stimuli signals) a predefined set of motions to the motion platform. Alternatively, the controller 120 can instruct the motion platform based on the input received by the input device 130. For example, the processor 16 is configured to instruct the controller 14 to cause execution of those motions for which expected information about a subject's perception of those motions would most contribute to improving an estimate of a subject's vestibular threshold. Such an estimate can be used to construct a vestibulogram, which shows the subject's vestibular threshold at different frequencies.

Referring back to FIG. 1, the controller 120 instructs the motion platform 110 to execute motions. For example, the motions can be selected for those motions for which expected information about the subject's perception of those motions would most contribute to improving an estimate of a subject's vestibular threshold.

Example of a Psychometric Function

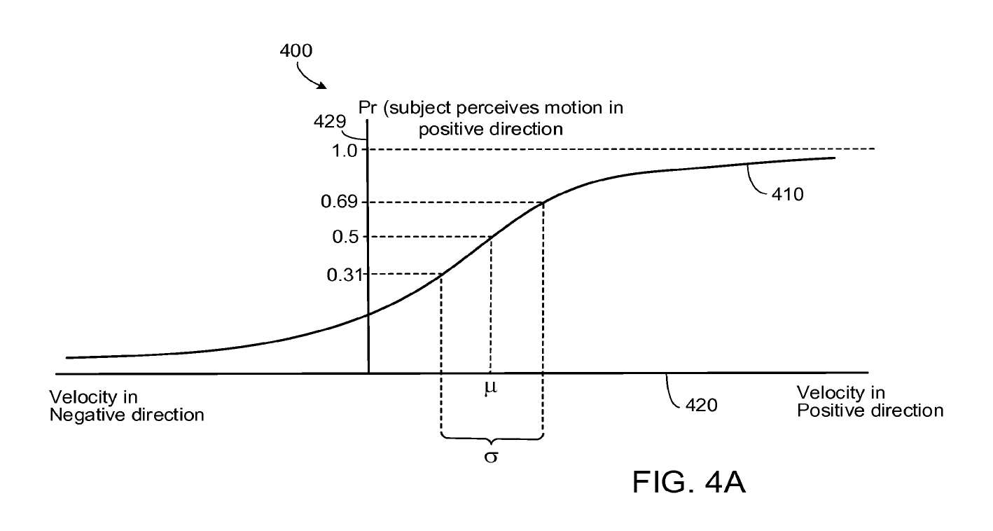

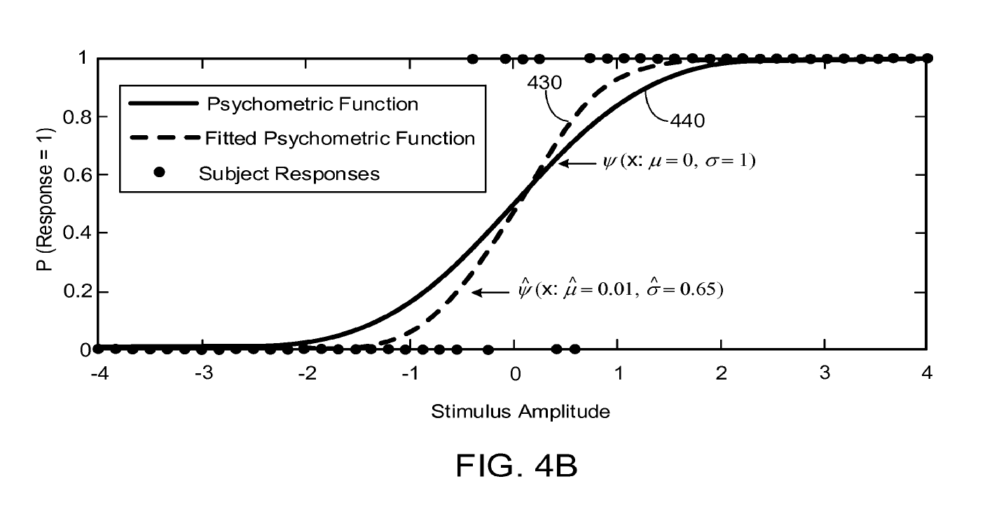

FIG. 4A shows an example of an estimated psychometric function 410 (e.g., vestibular function) fitted from data (e.g., binary responses) input from a subject 150. (Data points are not shown in FIG. 4A.) The psychometric function 410 can represent a probability that the subject 150 correctly perceives motion in a particular direction at a particular frequency. The horizontal axis 420 indicates the peak velocity of the motion profile experienced by the subject 150, with positive values indicating motion in one direction and negative values indicating motion in an opposite direction.

The vertical axis 429 indicates the estimated probability that, when subjected to motion in the positive or negative direction at a particular amplitude, the subject 150 will perceive motion in the positive direction as a function of that motion's peak velocity amplitude. As is apparent, when the motion is in a positive direction at relatively high amplitude, the subject has no difficulty perceiving it. Hence, the probability of correctly perceiving the motion approaches 1.0. In contrast, when the motion is in the negative direction at high velocity, the subject rarely makes the mistake of perceiving motion in the positive direction. Thus, the probability that the subject 150 will report positive motion given a high peak velocity in the negative direction would approach zero. At some amplitude in between, the probability that the subject 150 correctly identifies positive motion reaches 50%, thus indicating that the subject 150 can do no better than guessing. This amplitude, which is indicated on the horizontal axis 420 as μ, is a statistic that represents the vestibular “bias.” Another statistic, the “threshold,” or “spread,” which is shown as σ in FIG. 4A, represents the slope of the psychometric function 410 in the vicinity of the psychometric threshold.

In some examples, the psychometric function 410 is a fitted Gaussian probability density function given by:

$$Ψ ( x ; {b}_{1} , {b}_{2} ) = \frac{1}{2 π} {\int }_{- ∞}^{{b}_{1} {b}_{2} x} exp ( - {z}^{2} / 2 ) d z \qquad ( 3 )$$

For a series of responses by the subject 150, for example, at a single frequency, the procedure provides estimates of parameters μ and σ of the psychometric function 410, or equivalently, estimates of b₁, b₂ from which estimates of the parameters μ and σ can be derived. In either case, these estimates can have errors or uncertainties.

Accordingly, by measuring the psychometric function 410, a psychometric threshold σ for a specific type of motion, body orientation, and frequency can be estimated. Such measurements can be repeated for a range of frequencies, and a vestibulogram for a specific type of motion, body orientation, and frequency can be obtained from the resulting estimation for psychometric threshold σ.

The psychometric function 410 is traditionally determined using binary data obtained using standard discrimination tasks. The concepts described above can be generalized to a family of vestibulometric functions that characterize vestibular probabilities as a function of frequency. For example, as described in detail later, vestibulo-ocular measurements can be converted to a probability between 0 and 1 and plotted and fit in the same manner. Similarly, confidence ratings can also be plotted and fit in the same manner.

FIG. 4B shows another example of an estimate of a psychometric function 430 using a maximum likelihood Gaussian fit from a simulation of an experiment. Psychometric function 440 is the actual underlying psychometric function used in the simulation. The black dots in FIG. 4B indicates to a subject's binary response, where 0 corresponds to when the subject perceived a negative stimulus and 1 corresponds to when the subject perceived a positive stimulus.

General Methodology

According to the new methods described herein, a vestibular test is carried out with all of the motions aligned with gravity. For example, x-translation, y-translation, and z-translation can be performed in the down-back, side-up, up-right position, respectively. Such configurations of testing provide greater sensitivity in detecting vestibular dysfunctions. For example, the estimated threshold for such configurations can be compared to known thresholds for normal subjects of corresponding configurations. The test can include all x-, y-, z-translations with motions along gravity (Z axis).

A vestibular test can be carried out with motions that are aligned along gravity as well as motions that are not aligned with gravity. In this case, the two different types of motions can be compared to each other to estimate a subject's ability to sense motion.

Additionally, or separately, a vestibular test can obtain binary responses, VOR measures, or confidence ratings during the test, or a vestibular test can obtain any combination of the above measures.

These aspects are described in further detail below.

Estimating a Psychometric Function with Motion along Gravity

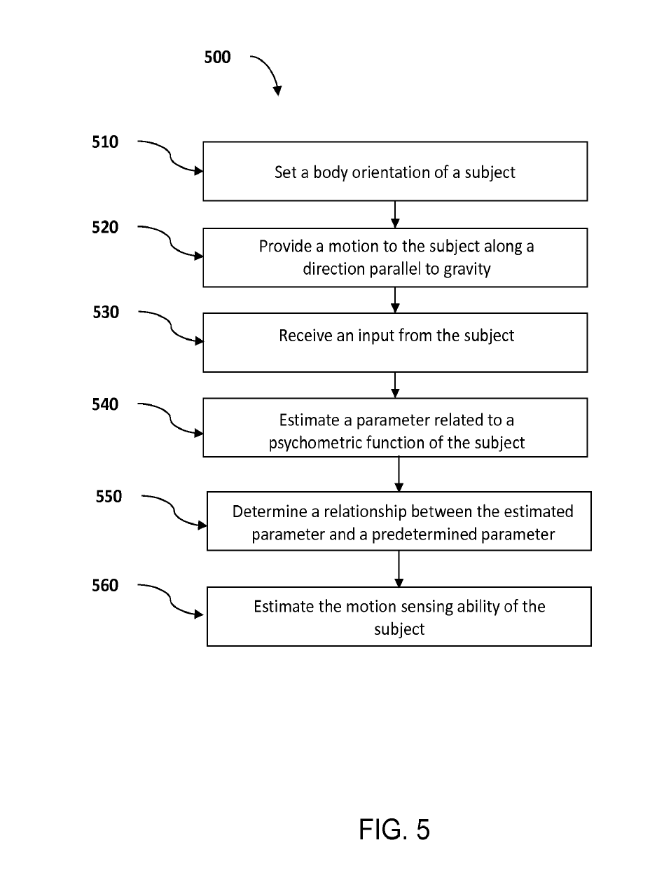

Referring to FIG. 5, a flow chart 500 depicts example operations for estimating a subject's motion sensing abilities. Operations include setting a body orientation of the subject 150 (510). This can be achieved by supporting the subject 150 on a motion platform 110, and orienting the motion platform 110. In some implementations, the subject 150 can be oriented in an up-right position (e.g., shown in FIG. 2a.) Alternatively, the subject 150 can be oriented in a side-up position (e.g., shown in FIG. 2b) or back-down position (e.g., shown in FIG. 2c).

Operations also include providing a motion to the subject 150 along a direction parallel to gravity (520). The motion can be provided by moving the motion platform 110. In some implementations, the subject 150 is the up-right position. In this case, the motion can be along the positive or negative z direction such that the motion is along a direction parallel to gravity. In some other implementations, where the subject 150 is in the side-up position, the motion can be along the positive or negative y direction such that the motion is along a direction parallel to gravity. In yet some other implementations, where the subject 150 is in the back-down position, the motion can be along the positive or negative x direction such that the motion is along a direction parallel to gravity. In some implementations, the motion may not need to be strictly parallel to gravity. The parallel alignment can be within 5 degrees (e.g., within 15 degrees, within 30 degrees) The motion may be considered parallel when having a major direction component along the direction parallel to gravity. For example, the largest direction component may be along the direction parallel to gravity.

An input from the subject 150 is received in operation (530), where the input is indicative of the subject's perception of the motion (520). For example, the input can include a binary response and/or a confidence rating. In some implementations, operations (520)-(530) can be one trial during the psychometric test. In some implementations, operations (520)-(530) can be repeated multiple times such that a set of binary responses and/or a set of confidence ratings are received.

At operation (540), a parameter related to a psychometric function of the subject 150 is estimated. In some implementations, the received binary response and/or confidence rating (or received set of binary responses and/or set of confidence ratings) in (530) can be used to estimate the parameter. For example, the parameter can be a psychometric threshold (σ) derived from the psychometric function. If the test is a vestibular test, the parameter can be a vestibular threshold derived from a vestibular function.

Operations further include determining a relationship between the estimated parameter and a predetermined parameter (550). The relationship can be a correlation (e.g., correlation function, magnitude comparison). In some implementations, the predetermined parameter is determined from estimating a corresponding parameter for a group of normal subjects. For example, operations (510)-(540) can be executed for a group (e.g., larger than 50, larger than 100, larger than 200) subjects with known normal motion sensing abilities. The estimated vestibular threshold for such a group of normal subjects can be considered as the predetermined parameter to be compared with the estimated parameter obtained for subject 150.

The motion sensing ability of the subject 150 is estimated in operation (560). The estimation can be based on the relationship in operation (550). In some implementations, the subject 150 can be evaluated to have a vestibular dysfunction when the estimated parameter is significantly greater (e.g., 5 times or larger, 10 times or larger, 15 times or larger, 20 times or larger) than the predetermined parameter. In some implementations, the subject 150 can be evaluated to be a malingerer when the estimated parameter is not statistically significantly greater than the predetermined parameter. For example, if a statistical probability, p, of a difference between the estimated parameter and the predetermined parameter, is less than an agreed upon statistical standard such as p<0.01, then the subject 150 can be evaluated to be a malingerer. Other example values of the statistical standard are p<0.005 or p<0.05.

In some implementations, the predetermined parameter can be an estimated parameter for the subject 150 during earth-horizontal motion.

Estimating a Psychometric Function for an Orientation of the Subject and Another Psychometric Function for Another Orientation of the Subject

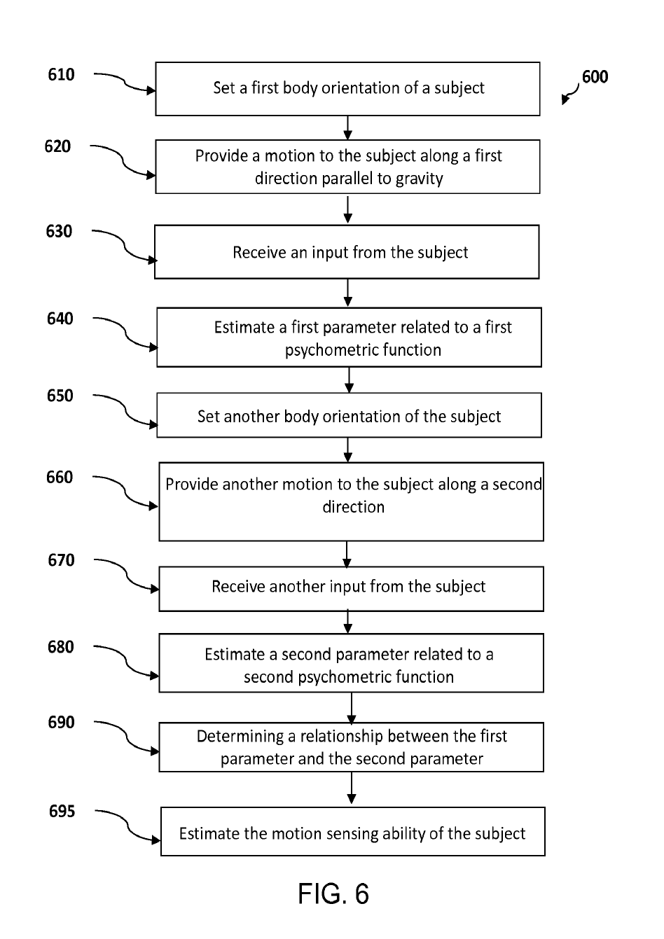

Referring to FIG. 6, a flow chart 600 depicts example operations for estimating a subject's motion sensing abilities. Operations include setting a first body orientation of the subject 150 (610), similar to (510).

Operations also include providing a motion to the subject 150 along a first direction parallel to gravity (620), similar to (520).

An input from the subject 150 is received in operation (630), where the input is indicative of the subject's perception of the motion (620). For example, the input can include a binary response and/or a confidence rating. In some implementations, operations (620)-(630) can be one trial during the psychometric test. In some implementations, operations (620)-(630) can be repeated multiple times such that a set of binary responses and/or VOR measurements and/or a set of confidence ratings are received. It is also understood that, even if the subject 150 provides a false perception, that input is considered indicative of the subject's perception of the motion.

Operations further include estimating a first parameter related to a first psychometric function (640), based on the input received in (630). The first psychometric function can be related to the type of motion (620). In some implementations, the received binary response and/or confidence rating (or received set of binary responses and/or set of confidence ratings) (630) can be used to estimate the first parameter. For example, the first parameter can be a first psychometric threshold (σ) derived from the first psychometric function. If the test is a vestibular test, the first parameter can be a first vestibular threshold derived from a first vestibular function.

At operation (650), another body orientation of the subject 150 is set. For example, if the body orientation in operation (610) is the up-right position, then the other body orientation can be set as the side-up position. Alternatively, if the body orientation in operation (610) is the side-up position, then another body orientation can be set as the up-right position. In some implementations, the body orientation can be set as the back-down position or front-down position.

Operations also include providing another motion to the subject along a second direction (660).

In some implementations, the second direction can be different from the direction of gravity. When the other motion is along the same direction as the motion (620) in head coordinates, due to the different body orientations, these two motions are along different directions in earth coordinates. For example, if the motion (620) is a z-translation in the upright position (which is an earth-vertical-translation with z-translation), then the other motion can be a z-translation in the side-up position (which is an earth-horizontal-translation with z-translation). As another example, if the motion (620) is a y-translation in the side-up position (which is an earth-vertical-translation with y-translation), then another motion can be a y-translation in the upright position (which is an earth-horizontal-translation with y-translation)

In some other implementations, the second direction can be parallel to the direction of gravity. The another motion and the motion in (620) are along the same direction in earth coordinates, but due to the different body orientations, these two motions are along different directions in head coordinates. For example, if the motion (620) is a z-translation in the up-right position (which is an earth-vertical-translation with z-translation), then the other motion can be a y-translation in the side-up position (which is an earth-vertical-translation with y-translation). As another example, if the motion (620) is a z-translation in the up-right position (which is an earth-vertical-translation with z-translation), then the other motion can be a x-translation in the back-down position (which is an earth-vertical-translation with x-translation).

Another input from the subject 150 is received in operation (670), where the input is indicative of the subject's perception of the motion (660). Similar to (630), the other input can include a binary response and/or a confidence rating. At operation (680), a second parameter related to a second psychometric function is estimated, based on the input received in (670). The second psychometric function can be related to the type of motion (660). In some implementations, the received binary response and/or confidence rating (or received set of binary responses and/or set of confidence ratings) in (630) can be used to estimate the second parameter. For example, the second parameter can be a second psychometric threshold (σ) derived from the second psychometric function. If the test is a vestibular test, the second parameter can be a second vestibular threshold derived from a second vestibular function.

In some implementations, the order of operations (610)-(640) and operations (650)-(680) can be reversed.

Operations also include determining a relationship (e.g., correlation) between the first parameter and the second parameter (690). For example, when the psychometric test is a vestibular test, the correlation can be between the first vestibular threshold and the second vestibular threshold. In some implementations, the operation (690) can include producing a first vestibulogram based on the first parameter and a second vestibulogram based on the second parameter. This can be achieved by measuring vestibular thresholds for a range of frequencies (e.g., 0.05 Hz-10Hz). Then the correlations can be between the first vestibulogram and the second vestibulogram. The correlation can provide a metric such as a correlation parameter (e.g., by calculating cross correlation) indicative of the degree of correlation between the first parameter and second parameter (or the first vestibulogram and the second vestibulogram). In this specification, correlation between two parameters can include a statistical comparison of two parameters or a comparison of magnitude.

The motion sensing ability of the subject 150 is estimated in operation (695), based on the relationship determined in operation (690). For example, the subject 150 can be estimated to have a vestibular dysfunction when the first parameter (which is measured along a first direction parallel to gravity) is 5 times or larger (e.g., 10 times or larger, 15 times or larger, 20 times or larger) than the second parameter (which was measured along a second direction different from the first direction).

In some implementations, the subject 150 can be estimated to be a malingerer based on the relationship in operation (690). For example, if the relationship indicates that thresholds for the first direction and the second direction are substantially similar (e.g., within 10%, within 30%, within 50% of each other) but the subject 150 expresses to have a vestibular dysfunction, the subject 150 can be evaluated to be a malingerer. In some examples, the subject 150 can be evaluated to be a malingerer when the first parameter for earth-vertical translations is not statistically significantly greater than the second parameter. For example, if a statistical probability, p, of a difference between the first parameter and the second parameter, is less than an agreed upon statistical standard such as p<0.01, then the subject 150 can be evaluated to be a malingerer. Other example values of the statistical standard are p<0.005 or p<0.05.

In some implementations, the first parameter can be used to produce a first vestibulogram and the second parameter can be used to produce a second vestibulogram. A determined relationship between the first vestibulogram and the second vestibulogram can be used to evaluate whether the subject has a vestibular dysfunction or is likely to be a malingerer. For example, if the relationship indicates that first vestibulogram and the second vestibulogram are statistically substantially different from the second parameter, the subject can be evaluated to have a vestibular dysfunction. For example, if a statistical probability, p, of a difference between the first vestibulogram and the second vestibulogram, is less than an agreed upon statistical standard such that p<0.01, then the subject 150 can be evaluated to be have a vestibular dysfunction. On the other hand, if the relationship indicates that first vestibulogram is not “statistically different from the vestibulogram parameter (e.g., p<0.01)”, but the subject 150 expresses to have a vestibular dysfunction, the subject 150 can be evaluated to be a malingerer. Other example values of the statistical standard are p<0.005 or p<0.05. In some implementations, the correlation can be calculated by a variety of methods including direction comparison, normalization, determining correlation functions.

Data Collection Including Confidence Ratings

Psychometric tests can be used to collect data including confidence ratings of a subject's perception of stimuli. The collected confidence ratings, which can be assigned to their corresponding stimuli, can be used to improve the quality of collected data and reduce testing time.

Conventionally, thresholds of a psychometric test are determined from a set of binary responses received from the subject 150. However, such approaches do not collect all available information of the subject's responses. For example, such approaches do not evaluate the subject's confidence of his or her binary responses. However, for some type of psychometric tests, adding a third option to the binary response can improve the quality of the results. The third option can be added by asking the subject 150 to choose one of three responses (e.g., “left”, “right”, or “uncertain”) instead of just one of two responses (e.g., “left” and “right”). Then the collected responses can be analyzed using an indecision model such as a three-option model. However, in this approach, the conventional binary response detection analysis cannot be applied due to the additional “uncertain” response.

The disclosed techniques can be used to collect data including confidence ratings such that both the conventional binary detection analysis and indecision analysis can be applied to the collected data.

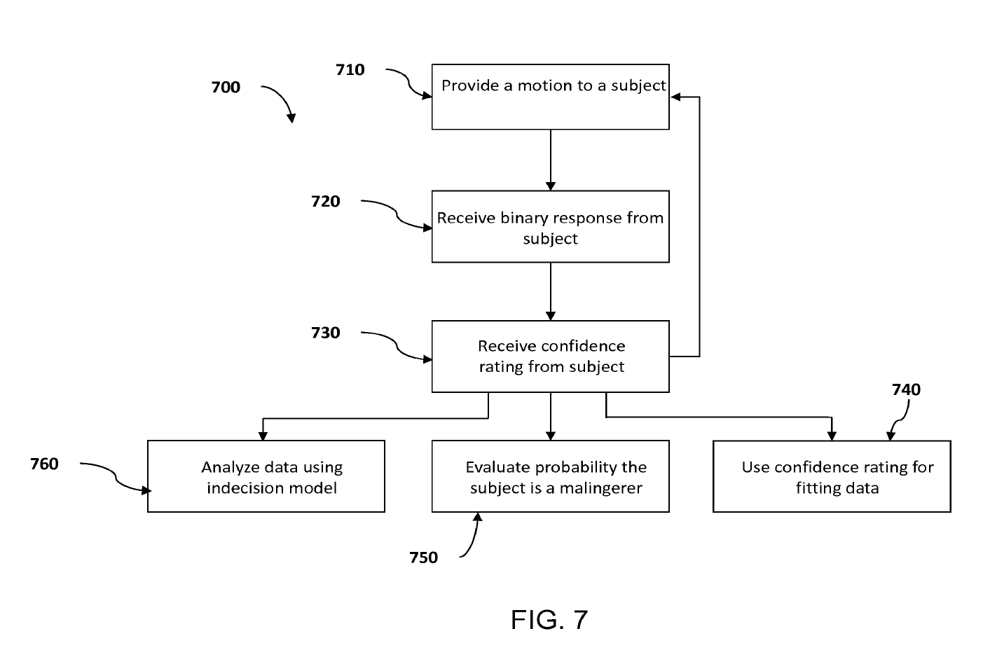

Referring to FIG. 7, a flow chart 700 depicts example operations for receiving confidence ratings. Operations include providing a motion to a subject 150 (710). In some implementations, the motion can include any of x, y, z translation, roll, pitch, or roll rotation. For example, the z translation can be executed when the subject 150 is in the upright position such that the motion is parallel to the direction of gravity.

Operations also include receiving a binary response from the subject 150 through an input device 130 (720). The binary response represents the motion perceived by the subject 150 regarding the motion provided in (710). For example, when the provided motion is a positive translation in the y direction (which is the “left” direction in FIG. 1), the subject can either input a binary response corresponding to “left” or “right”. In this example, if the subject 150 inputs “right” the binary response is incorrect and if the subject inputs “left” the binary response is correct.

Operations further include receiving a confidence rating from the subject 150 through the input device 130 (730). The confidence rating represents how confident the subject 150 is regarding the binary response input in (720). As such, during this operation, the subject 150 can provide an assessment of his or her confidence regarding the perceived motion of (710).

The confidence rating can be in any of the following form: (1) a quasi-continuous rating (e.g., 50% confidence to 100% confidence in 1% increments); (2) a binary rating (e.g., “guessing” versus “certain”); (3) a quinary rating (e.g., 1 to 5 where 1 is “guessing” and 5 is “certain”) or a N-level discrete rating (e.g., 1 to N where 1 is “guessing” and N is “certain”); or (4) a wagering rating. For example, when the “quasi-continuous rating” is used, the subject 150 can input his or her confidence rating as a percentage value regarding the binary response input in (720). The input confidence rating can be communicated to a processor 140, which can estimate a psychometric function or threshold of the test based on the received data.

In some implementations, operations (710)-(730) can correspond to one trial during the test. The order of operations (720) and (730) can be reversed or occur simultaneously.

Further operations can be in included in process 700. In some implementations, the following operations can be executed for data (e.g., binary response, confidence rating) obtained from a single trial or data obtained from a plurality of trials. In other words, operations (710)-(730) can be executed once or multiple times before proceeding to the following operations. For each trial, there can be at least one binary response and at least one corresponding confidence rating.

Operations can also include using the received confidence rating during data collection (e.g., for each trial) and/or after data collection is complete (e.g., for multiple trials) for fitting data (740). This can improve the efficiency of the test and fit quality of an estimated psychometric function, which can estimated either from the binary responses, the confidence ratings, or both. In some implementations, the received confidence ratings can be fit with a cumulative distribution function (e.g., Gaussian cumulative distribution) to provide information on the point of subjective equality (PSE) and/or the width of the distribution (e.g., “sigma”) of the estimated psychometric function. For example, an indication that the subject moved in a positive direction with 83% confidence could be equivalent to a probability level of 0.83 on a psychometric function that varies between 0 and 1. An indication that the subject moved in a negative direction with 83% confidence could be equivalent to a probability level of 0.17 for that same psychometric function. Such fits can be useful for determining the parameter (e.g., amplitude, direction, frequency) of the stimulus signal (also may be referred as “stimulus”) for the next trial. In other words, the next stimulus can be adapted based on the received confidence rating from the subject 150.

In some implementations, the confidence ratings can be useful for estimating the psychometric function when the number of trials (M) is 35 or less (e.g., 30 or less, 25 or less, 20 or less, 15 or less). This is because for such a low number of trials (e.g., M<25), estimation of the psychometric using only binary responses yields large variability in the fit parameters. For example, a psychometric function estimated from 25 binary responses (from 25 trials) can have a standard deviation of the estimated width parameter (e.g., sigma or σ) to be 50% of the actual value of the width parameter. In other words, for a given M number of trials, fitting accuracy of confidence ratings can be higher than the fitting accuracy of binary responses. In some implementations, the fitting of confidence ratings and binary response can be combined to improve the accuracy. The received confidence ratings can be used in a closed-loop manner for estimating the psychometric function and its threshold.

Accordingly, data collection of confidence ratings can be used to improve the testing efficiency because: (1) a useful stimulus for the next trial can be determined; and/or (2) estimation of the psychometric function can be improved with a small number of trials. In some implementations, the received confidence ratings can provide additional data to validate or invalidate the binary response detection model or the indecision model for different classes of patients.

Operations may include evaluating the probability that the subject is a malingerer based on the received binary responses and confidence ratings (750). In some implementations, evaluating includes correlating the received set of binary responses to the corresponding set of received confidence ratings. The correlation can provide a correlation parameter (e.g., by calculating correlation functions) indicative of the degree of correlation between the received binary responses and the confidence ratings. For example, if the calculated correlation parameter is different (e.g. smaller) from a predetermined correlation threshold, then the subject 150 can be evaluated to be a malingerer. The reason is, if the subject 150 is faking his or her binary responses, it is difficult to also fake his or her confidence ratings such that it shows the expected correlation with his or her fake binary responses. The expected correlation threshold can be determined by calculating the correlation parameter of known normal subjects 150 and/or via simulations.

Operations may include analyzing the received binary response and confidence rating using an indecision model (760). In some implementations, the confidence rating can be used to re-label its corresponding binary response (e.g., “left” or “right”) as “uncertain” when the confidence rating is below a confidence threshold. For example, when using a quasi-continuous rating (50% confidence to 100% confidence), the confidence threshold can be set as 55%. In this example, the binary response with confidence rating below 55% can be considered as a guess and re-labeled as “uncertain”. As a result, the modified binary responses can include the three options including a binary response (e.g., “left” or “right”) and “uncertain”. Such modified binary responses can be analyzed using the indecision model.

Alternatively, in some implementations, the confidence ratings can be used to eliminate binary responses where it is determined that the subject 150 has simply guessed in providing the binary response. For example, when using the quasi-continuous rating (50% confidence to 100% confidence), if a certain binary response has a confidence rating below the confidence threshold (e.g., set as 55%), that binary response can be eliminated from the collected data. As a result, the binary response still includes two options (e.g., “left” or “right”), but the resulting number of binary responses may be reduced due to elimination. Although, the number of binary responses is reduced, the quality of data can improve because only responses that were not guesses were analyzed.

In some implementations, the operations of process 700 can be applied to other types of psychometric tests than vestibular tests. For example, the psychometric tests can be visual tests where the stimuli are visual cues instead of motion.

Data Collection Including VOR

In some implementations, the VOR can be measured during vestibular tests. This can be achieved by adding a device (e.g., video system, search coil system) for measuring eye position during the procedures that estimate a subject's perceptual threshold. In particular, VOR thresholds can have strong correlation to perceptual thresholds at frequencies above about 1 Hz. In some implementations, an input device 130 can include the device for measuring the eye position.

VOR data can used in the above operations (e.g., (710)-(760)), along with confidence ratings (or instead of confidence ratings). This is because the VOR can have a correlation to confidence ratings. Large motion stimuli can induce VOR responses that are large relative to the VOR variations at rest (“noise”) above the VOR threshold. In contrast, small motion stimuli can induce VOR responses that are small relative to the VOR at rest. For example, if one measures the VOR at rest to have a standard deviation of 1 deg and then provide a motion that yields a +1 deg VOR response, this can yields a signal to noise ratio of 1 because the amplitude of the VOR equals the standard deviation at rest. This can be showed to be equivalent to a vestibulometric probability of 0.8413 because the cumulative distribution function equals 0.8413 when the ratio of signal (VOR evoked by motion) to noise (VOR at rest) equals 1. As another example, a VOR of −2 can be shown to be equivalent to a vestibulometric probability of 0.0228 because the cumulative distribution function equals 0.0228 when the ratio of signal (VOR evoked by motion) to noise (VOR at rest) equals −2.

In some implementations, the VOR can be useful for estimating a cumulative distribution function related to a psychometric function when the number of trials (M) is 35 or less (e.g., 30 or less, 25 or less, 20 or less, 15 or less). This is because for such a low number of trials (e.g., M<25), estimation of the psychometric using only binary responses yields large variability in the fit parameters. For example, a psychometric function estimated from 25 binary responses (from 25 trials) can have a standard deviation of the estimated width parameter (e.g., sigma or σ) to be 50% of the actual value of the width parameter. In other words, for a given M number of trials, cumulative distribution fitting accuracy of VOR can be higher than the fitting accuracy of binary responses. In some implementations, the fitting of VOR and binary response can be combined to improve the accuracy. Analogous to confidence rating described above, the measured VOR can be used in a closed-loop manner for estimating the psychometric function and its threshold.

Accordingly, VOR data collection can be used to improve the testing efficiency because: (1) a useful stimulus for the next trial can be determined; and/or (2) estimation of a psychometric function can be improved with a small number of trials. In some implementations, the received VORs can provide additional data to validate or invalidate the binary response detection model or the indecision model for different classes of patients.

In some implementations, the measured VOR can be used in a closed-loop manner for estimating a vestibularmetric function and its threshold, in a similar manner as described above.

In some implementations, the probability that the subject is a malingerer can be evaluated based on the received binary responses and VORs. The evaluation can include correlating the received binary responses and the VORs. The correlation can provide a correlation parameter (e.g., by calculating correlation functions) indicative of the degree of correlation between the received binary responses and VORs. For example, if the calculated correlation parameter is different (e.g. smaller) from a predetermined correlation threshold, then the subject 150 can be evaluated to be a malingerer. The reason is, if the subject 150 is faking his or her binary responses, it is difficult to also fake his or her VOR such that it shows the expected correlation with his or her fake binary responses. The expected correlation threshold can be determined by calculating the correlation parameter of known normal subjects 150 and/or via simulations.

Because the subject 150 cannot control this involuntary VOR at frequencies above 1 Hz (e.g., above 2 Hz, above 3 Hz), the VOR measurement provides additional information, which can be correlated with the psychometric function estimated from binary responses. A mismatch at high frequencies (e.g., 1-2 Hz) between the subject's psychometric function relating to his or her perceptual threshold and the subject's VOR responses can indicate malingering. In other words, the level of statistical significance of the difference from normal would provide a probability that the subject 150 is a malingerer. This approach can be combined with the techniques relating to measuring confidence ratings.

Accordingly, in some implementations, the disclosed techniques relate to an input device 130 that can receive a reference response (e.g., confidence rating, VOR). It is also understood that the input device 130 can receive an input set which includes any combination of a binary response, a confidence rating, and a VOR.

Increasing the Difficulty of Trials to Increase Sensitivity

Psychometric tests can include factors than increase the difficulty of a trial during the test. In some implementations, if the individual trials are made more difficult or complex, the test becomes more sensitive. The increased sensitivity can improve the accuracy of tests and reduce the overall testing time, and thereby be helpful in evaluating whether the subject has a disorder or is likely to be a malingerer.

In some implementations, an individual trial involving a motion stimulus can be made more difficult by providing a distracting motion prior to the motion stimulus. The distracting motion can be provided, for example, in a direction different than that of the motion stimulus. The distracting motions can be of variable amplitude and frequency, as compared to the motion stimulus. The motion stimulus can be preceded by vibrations, exposure of light, hearing tasks, cognitive tasks to make the vestibular test harder for the subject. In some implementations, an individual trial can be made more difficult by asking the subject to include in the response, a confidence rating associated with the subject's perception of the stimuli. In some implementations, various aspects of the disclosed techniques can be combined.

### Applications of the New Methods

Determining Vestibular Disabilities

The disclosed techniques can be used to evaluate whether a subject 150 has a vestibular disability. In some implementations, evaluations can be based on vestibular tests including motions aligned along gravity and motions that are not aligned along gravity. This is because patients (e.g., subjects who have vestibular dysfunctions) can have difficulties sensing motions aligned along gravity than motions that are not aligned along gravity. For example, patients with impaired vestibular systems can finding sensing up/down motions (along gravity) to be more difficult than sensing left/right motions (perpendicular to gravity). On the other hand, normal subjects may not find substantial differences in sensing these two types of motions. Therefore, the difference between patients and normal subjects would be greater when the motion is aligned with gravity.

In some other implementations, evaluations can be based on vestibular tests including motions aligned along gravity at different body orientations. This is because patients can have different levels of sensitivity for motions along gravity but with different body orientations. For example, patients can find sensing left/right motions (along gravity) to be more difficult than sensing up/down motions (along gravity). On the other hand, normal subjects may not find substantial differences in sensing these two types of motions.

Detecting Malingerers

The disclosed techniques can be used to evaluate whether a subject 150 is a malingerer in vestibular tests. In some implementations, evaluations can be based on comparing thresholds at a frequency or vestibulograms between motions aligned along gravity and motions that are not aligned along gravity. For such comparisons, patients may show much larger differences than normal subjects. Malingerers are unlikely to show such large deviations, because they may not perceive substantial differences in sensing these two types of motions.

In some other implementations, evaluations can be based on comparing thresholds at a frequency or vestibulograms between motions aligned along gravity but for different body orientations. For such comparisons, patients may show much larger differences than normal subjects. Malingerers are unlikely to show such large deviations, because they may not perceive substantial differences in sensing these two types of motions.

In another aspect, confidence ratings correlate with amplitudes of stimuli. A psychometric function relates the correlation between received binary responses and amplitudes of stimuli. Thus, normally, confidence ratings should correlate with binary responses of the subject 150. However, were the subject 150 faking his or her binary responses (in other words, malingering), the correlation between confidence ratings and binary response can be low. For example, normally, confidence ratings should be high for large stimuli because the subject 150 is more likely to confidently perceive the stimuli. Similarly, confidence ratings should be low for small stimuli. If the correlation between confidence ratings and the binary response differ from this trend, this would be an indication that the subject 150 is malingering. Accordingly, the level of correlation indicates the probability that the subject 150 is a malingerer.

Randomness can also be included in setting the amplitudes of the stimuli. In this case, confidence ratings and binary responses received by the subject 150 should also exhibit the randomness. If it were that the confidence ratings and/or the binary response lacked randomness or if the confidence ratings and the binary responses did not share the expected correlation, these would indicate that the subject 150 is faking his or her responses. This is because it is difficult for the subject 150 to generate inputs approximating random sequences voluntarily.

Data Collection Including Confidence Ratings

The disclosed techniques can be useful in clinical trials involving forced-choice procedures such as measuring binary responses. Conventional methods force an input from a subject 150 to be either: (1) a binary response (e.g., yes/no, left/right, or up/down); or (2) a three option response (e.g., yes/no/uncertain, left/right/uncertain, or up/down/uncertain). If a binary response is chosen, one cannot apply an indecision analysis. If a three option response is chosen, one cannot apply a conventional binary response analysis. However, the disclosed techniques enable collection of data for both analyses. This is because both binary responses and confidence ratings can be collected. In addition, the conventional binary response detection analysis can be improved by eliminating data (e.g., binary response) which are considered to be guesses. After elimination, the entire data can be fitted using conventional binary response detection analysis with improved estimation of the psychometric function.

Moreover, the option of being able to analyze in either binary response detection analysis or indecision analysis is advantageous because certain psychometric tests can be analyzed with higher quality in either analysis but not both. In some cases, it is unclear which approach is better unless the actual data is measured and analyzed. However, this is not a concern in the disclosed techniques because either analysis is applicable. This aspect is important in psychometric tests which can involve many trials and be time consuming In addition, the additional information provided by collected confidence ratings can benefit from new types of analysis.

As an example, the disclosed techniques can streamline collection of data in vestibular diagnostic devices. The data collected can further improve accuracy or directly aid a diagnosis or help detect malingerers.

Overview of a Computing Device with a Processor

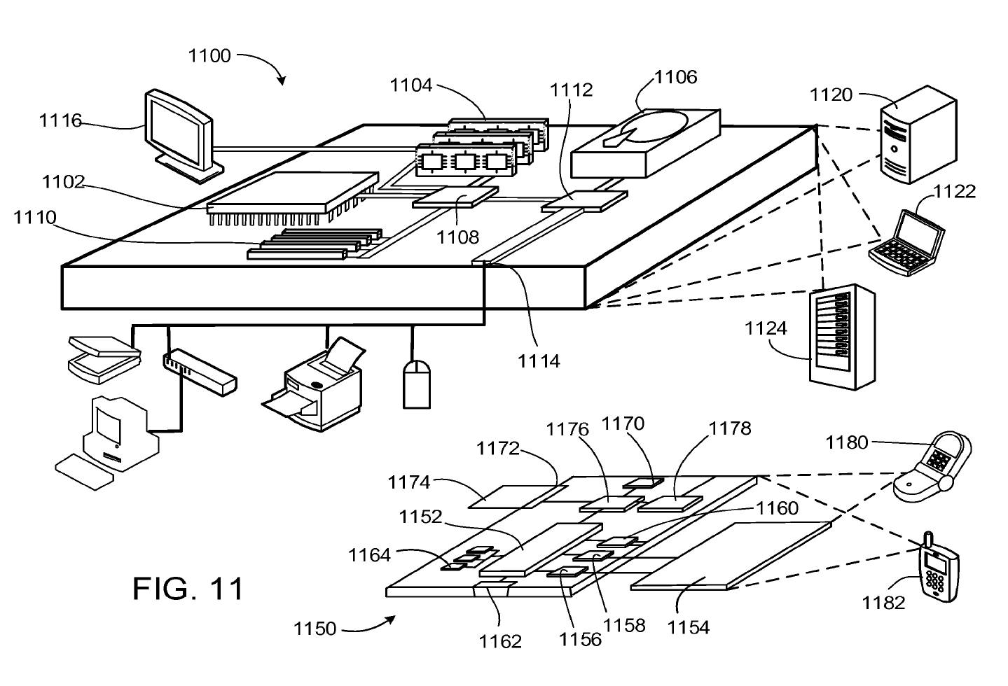

FIG. 11 shows an example of a computing device 1100 and a mobile device 1150, which may be used with the techniques described here. Computing device 1100 is intended to represent various forms of digital computers, such as laptops, desktops, workstations, personal digital assistants, servers, blade servers, mainframes, and other appropriate computers. Computing device 1150 is intended to represent various forms of mobile devices, such as personal digital assistants, cellular telephones, smartphones, and other similar computing devices. The components shown here, their connections and relationships, and their functions, are meant to be examples only, and are not meant to limit implementations of the techniques described and/or claimed in this document.

Computing device 1100 includes a processor 1102, memory 1104, a storage device 1106, a high-speed interface 1108 connecting to memory 1104 and high-speed expansion ports 1110, and a low speed interface 1112 connecting to low speed bus 1114 and storage device 1106. Each of the components 1102, 1104, 1106, 1108, 1110, and 1112, are interconnected using various busses, and may be mounted on a common motherboard or in other manners as appropriate. The processor 1102 can process instructions for execution within the computing device 1100, including instructions stored in the memory 1104 or on the storage device 1106 to display graphical information for a GUI on an external input/output device, such as display 1116 coupled to high speed interface 1108. In other implementations, multiple processors and/or multiple buses may be used, as appropriate, along with multiple memories and types of memory. Also, multiple computing devices 1100 may be connected, with each device providing portions of the necessary operations (e.g., as a server bank, a group of blade servers, or a multi-processor system). In some implementations the computing device can include a graphics processing unit.

The memory 1104 stores information within the computing device 1100. In one implementation, the memory 1104 is a volatile memory unit or units. In another implementation, the memory 1104 is a non-volatile memory unit or units. The memory 1104 may also be another form of computer-readable medium, such as a magnetic or optical disk.

The storage device 1106 is capable of providing mass storage for the computing device 1100. In one implementation, the storage device 1106 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 1104, the storage device 1106, memory on processor 1102, or a propagated signal.

The high speed controller 1108 manages bandwidth-intensive operations for the computing device 1100, while the low speed controller 1112 manages lower bandwidth-intensive operations. Such allocation of functions is an example only. In one implementation, the high-speed controller 1108 is coupled to memory 1104, display 1116 (e.g., through a graphics processor or accelerator), and to high-speed expansion ports 1110, which may accept various expansion cards (not shown). In the implementation, low-speed controller 1112 is coupled to storage device 1106 and low-speed expansion port 1114. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 1100 may be implemented in a number of different forms, as shown in the figure. For example, it may be implemented as a standard server 1120, or multiple times in a group of such servers. It may also be implemented as part of a rack server system 1124. In addition, it may be implemented in a personal computer such as a laptop computer 1122. Alternatively, components from computing device 1100 may be combined with other components in a mobile device, such as the device 1150. Each of such devices may contain one or more of computing device 1100, 1150, and an entire system may be made up of multiple computing devices 1100, 1150 communicating with each other.

Computing device 1150 includes a processor 1152, memory 1164, an input/output device such as a display 1154, a communication interface 1166, and a transceiver 1168, among other components. The device 1150 may also be provided with a storage device, such as a microdrive or other device, to provide additional storage. Each of the components 1150, 1152, 1164, 1154, 1166, and 1168, are interconnected using various buses, and several of the components may be mounted on a common motherboard or in other manners as appropriate.

The processor 1152 can execute instructions within the computing device 1150, including instructions stored in the memory 1164. The processor may be implemented as a chipset of chips that include separate and multiple analog and digital processors. The processor may provide, for example, for coordination of the other components of the device 1150, such as control of user-interfaces, applications run by device 1150, and wireless communication by device 1150.

Processor 1152 may communicate with a user through control interface 1158 and display interface 1156 coupled to a display 1154. The display 1154 may be, for example, a TFT LCD (Thin-Film-Transistor Liquid Crystal Display) or an OLED (Organic Light Emitting Diode) display, or other appropriate display technology. The display interface 1156 may comprise appropriate circuitry for driving the display 1154 to present graphical and other information to a user. The control interface 1158 may receive commands from a user and convert them for submission to the processor 1152. In addition, an external interface 1162 may be provide in communication with processor 1152, so as to enable near area communication of device 1150 with other devices. External interface 1162 may provide, for example, for wired communication in some implementations, or for wireless communication in other implementations, and multiple interfaces may also be used.

The memory 1164 stores information within the computing device 1150. The memory 1164 can be implemented as one or more of a computer-readable medium or media, a volatile memory unit or units, or a non-volatile memory unit or units. Expansion memory 1174 may also be provided and connected to device 1150 through expansion interface 1172, which may include, for example, a SIMM (Single In Line Memory Module) card interface. Such expansion memory 1174 may provide extra storage space for device 1150, or may also store applications or other information for device 1150. Specifically, expansion memory 1174 may include instructions to carry out or supplement the processes described above, and may include secure information also. Thus, for example, expansion memory 1174 may be provide as a security module for device 1150, and may be programmed with instructions that permit secure use of device 1150. In addition, secure applications may be provided via the SIMM cards, along with additional information, such as placing identifying information on the SIMM card in a non-hackable manner

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 1164, expansion memory 1174, memory on processor 1152, or a propagated signal that may be received, for example, over transceiver 1168 or external interface 1162.

Device 1150 may communicate wirelessly through communication interface 1166, which may include digital signal processing circuitry where necessary. Communication interface 1166 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1168. In addition, short-range communication may occur, such as using a Bluetooth, WiFi, or other such transceiver (not shown). In addition, GPS (Global Positioning System) receiver module 1170 may provide additional navigation- and location-related wireless data to device 1150, which may be used as appropriate by applications running on device 1150.

Device 1150 may also communicate audibly using audio codec 1160, which may receive spoken information from a user and convert it to usable digital information. Audio codec 1160 may likewise generate audible sound for a user, such as through a speaker, e.g., in a handset of device 1150. Such sound may include sound from voice telephone calls, may include recorded sound (e.g., voice messages, music files, and so forth) and may also include sound generated by applications operating on device 1150.

The computing device 1150 may be implemented in a number of different forms, as shown in the figure. For example, it may be implemented as a cellular telephone 1180. It may also be implemented as part of a smartphone 1182, personal digital assistant, tablet computer, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well. For example, feedback provided to the user can be any form of sensory feedback (e.g., visual feedback, auditory feedback, or tactile feedback). Input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user-interface or a Web browser through which a user can interact with an implementation of the systems and techniques described here), or any combination of such back end, middleware, or front end components. The components of the system can be interconnected by any form or medium of digital data communication (e.g., a network). Examples of networks include a local area network (“LAN”), a wide area network (“WAN”), and the Internet.

The computing system can include clients and servers. A client and server are generally remote from each other and typically interact through a communication network such as the network. The relationship of client and server arises by virtue of computer programs running on the respective computers and having a client-server relationship to each other.

### EXAMPLES

The methods and systems described herein are further illustrated using the following examples, which do not limit the scope of the claims.

Results 1—Vestibular Tests of Patients and Normal Subjects

Subjects

Vestibular tests were carried out for three patients who had undergone bilateral surgical ablation of both inner ears for bilateral vestibular schwannomas associated with neurofibromatosis type 2. The three patients had no residual vestibular function. All three patients were deaf and used an auditory brainstem implant during testing. Vestibular tests were also carried out for fourteen normal subjects (nine females and five males.) The mean age of the subjects (patients and normal subjects) were about 36.

Testing Method

To provide a broad assessment of vestibular functions, vestibular thresholds were measured for four different motions: (1) yaw rotation; (2) earth-vertical z-translation; (3) earth-horizontal y-translation; and (4) roil rotation. All motions were carried out in the up-right body orientation. Vestibular thresholds were measured as a function of frequency (e.g., 0.5-10 Hz.) Motion stimuli were generated using a MOOG 6DOF motion platform. Single cycles of sinusoidal acceleration were applied. The peak acceleration, peak velocity and total lateral displacement were proportional to one another.

At each onset of a motion stimulus a brief low pitch tone was sounded. After the motion stimulus was provided, a brief high pitch sound indicated that the subject should respond. Each subject was instructed to push the button in the left hand if the subject perceived a leftward (or downward) motion or to push the button in the right hand for rightward (or upward) motion. The subjects made a guess were the perceived motion was uncertain. The subjects were seated in a chair with a five-point harness in an upright position.

Each frequency was tested in a block of trials before switching to another frequency. All four different motions were tested at frequencies between 0.3 Hz and 10 Hz. Patients could only complete testing at the highest frequencies for some motion conditions. For normal subjects who mostly completed testing at all frequencies, testing took 10-12 hrs. For total loss patients who could not complete tests at lower frequencies, testing took 6-8 hrs.

Analysis Method

A hybrid approach was used to estimate psychometric functions. The hybrid approach included an adaptive three-down/one-up staircase that set the stimulus amplitude for each trial, with a maximum likelihood fit of the data. The maximum likelihood fit was performed using a generalized linear model (GLM). Direction of motion stimuli (e.g., left or right) was randomized. The data included a peak angular velocity amplitude vector and a binary response. After each trial, the GLM fit was performed. Data collection for each subject was terminated when the estimated standard deviation of the spread parameter was <20%. On average, 70-80 trials were used to obtained the desired confidence of variation.

Test Results

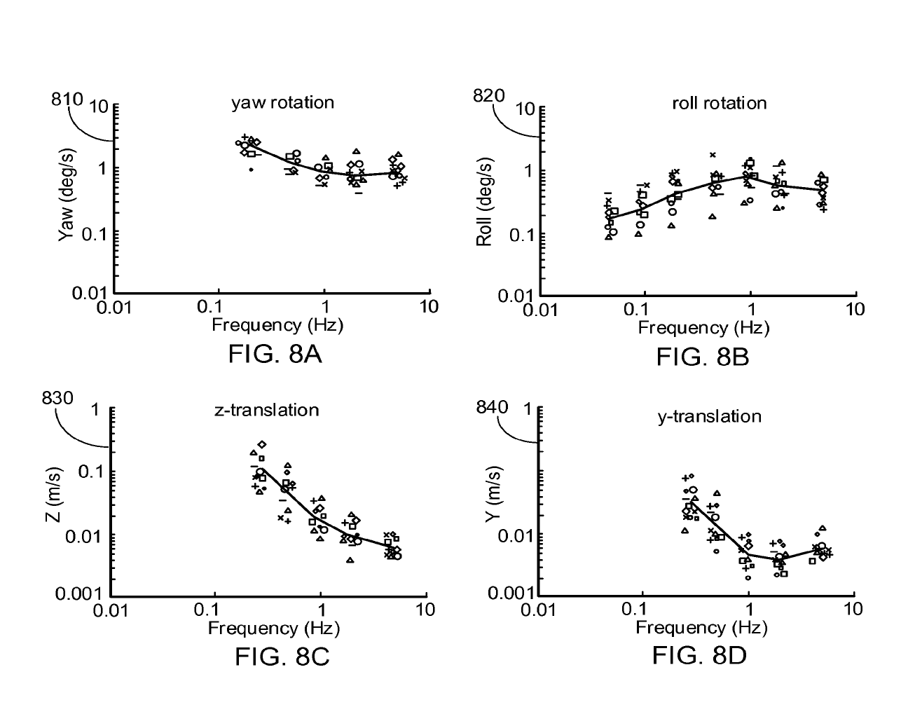

FIGS. 8A to 8D are a series of plots 810-840 showing peak stimulus velocity at the threshold as a function of frequency for the normal subjects. Different symbols correspond to different normal subjects. Plots 810, 820, 830 and 840 show data for yaw rotation, roll rotation, z-translation, and y-translation, respectively. Each data set for the four different motions indicates a low slope “plateau” region at high frequencies. Data for yaw rotation, z-translation, y-translation indicate that the thresholds substantially increase at lower frequencies, while data for roll rotation indicate that the thresholds substantially decrease.

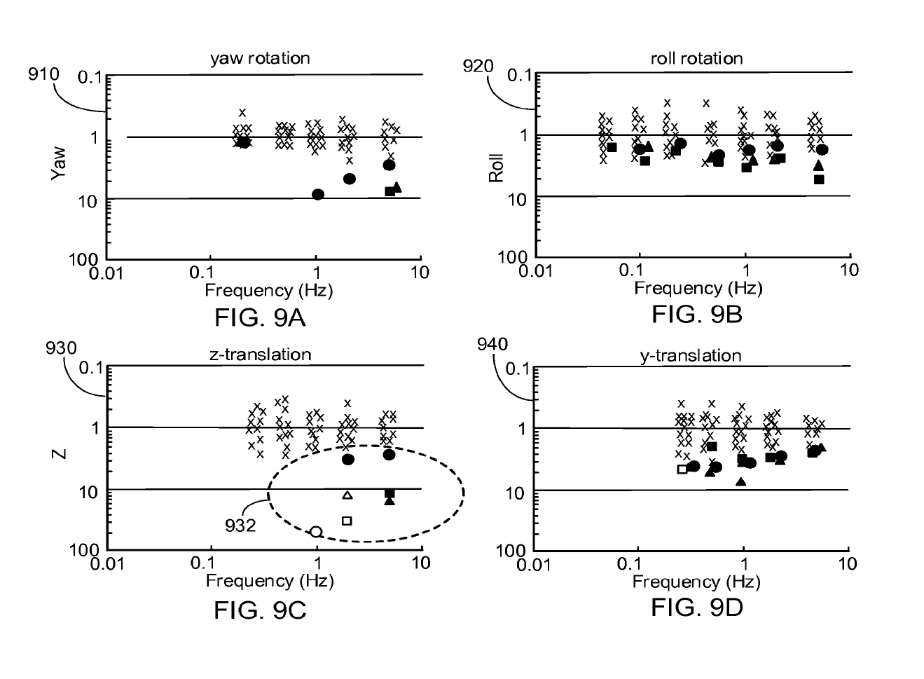

FIGS. 9A to 9D shows a series of plots 910-940 showing patient data normalized by geometric mean of data from the normal subjects at each frequency. The cross symbols correspond to data from normal subjects and the circle, square and triangle symbols correspond to data from the three patients. Plots 910, 920, 930 and 940 show data for yaw rotation, roll rotation, z-translation, and y-translation, respectively. The patients' data for yaw rotation and z-translation indicates that thresholds were substantially greater than that of the normal subjects where p-value<0.01. The three patients could not complete the test at low frequencies because motions needed to assay patient thresholds were beyond motion limits of the testing chair. The patients' data for roll rotation and y-translation indicated that thresholds showed an increase (p-value<0.01) compared to that of the normal subjects.

The results showed that the vestibular thresholds for the patients were higher than the thresholds for normal subjects. Note that patient deficits (i.e., threshold increases) showed up as downward shifts in the threshold relative to normal, which matches the standard practice for plotting audiograms which show hearing deficits in the same manner. The average thresholds for patients were at least 30% larger than the average for normal subjects for each frequency and for each type of motion. In addition, the vestibular thresholds for the patients were much higher for the motions of yaw rotation and z-translation than that for y-translation and roll rotation.

In plot 930, the square and triangle indicated within dash circle 932 correspond to the measured thresholds of two patients. In plot 930, the triangle, circle, and square data points indicated by dash circle 932 correspond to data from the patients. These patients' thresholds shown in 932 were greater than the thresholds for the normal subjects.

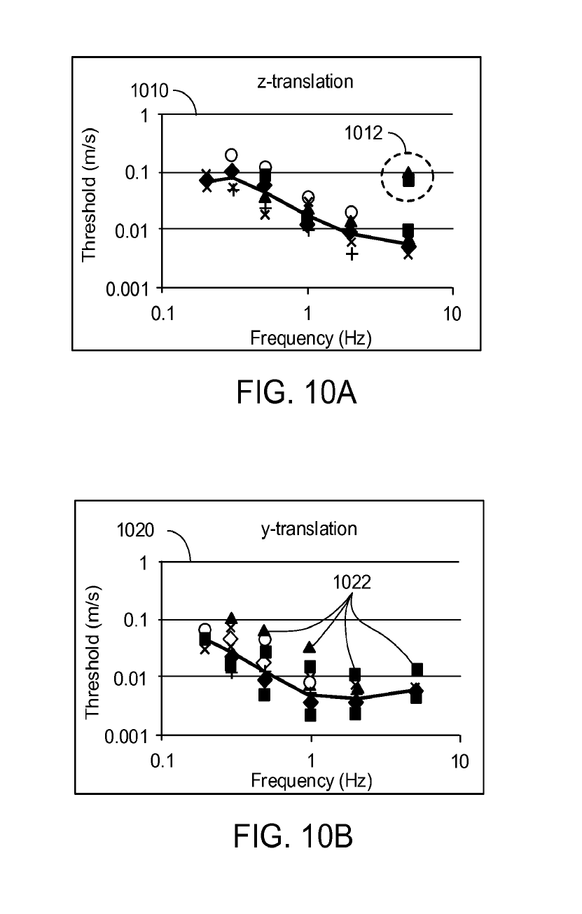

FIGS. 10A and 10B shows two plots 1010 and 1020 showing peak stimulus velocity at the vestibular threshold for patients and normal subjects. Plots 1010 and 1020 show data for z-translation and y-translation, respectively. Plots 1010 and 1020 are related to plot 930 and 940, respectively, but without the normalization by geometric mean data from the normal subjects. In plot 1010, the square and triangle indicated within dash circle 1012 correspond to the measured thresholds of two patients. In plot 1020, the triangle and square data indicated by 1022 correspond to data from the patients. These patients' thresholds shown in plot 1012 were about more than 10 times greater than the thresholds for the normal subjects. In contrast, plot 1020 indicates that for y-translation, thresholds of the patients were higher than that of the normal subjects by less than 10 times. Accordingly, the results show that the z-translation, which motion is along gravity, has higher sensitivity to evaluate a subject's motion sensing ability.

Results 2 —Increasing Difficulty of Trials

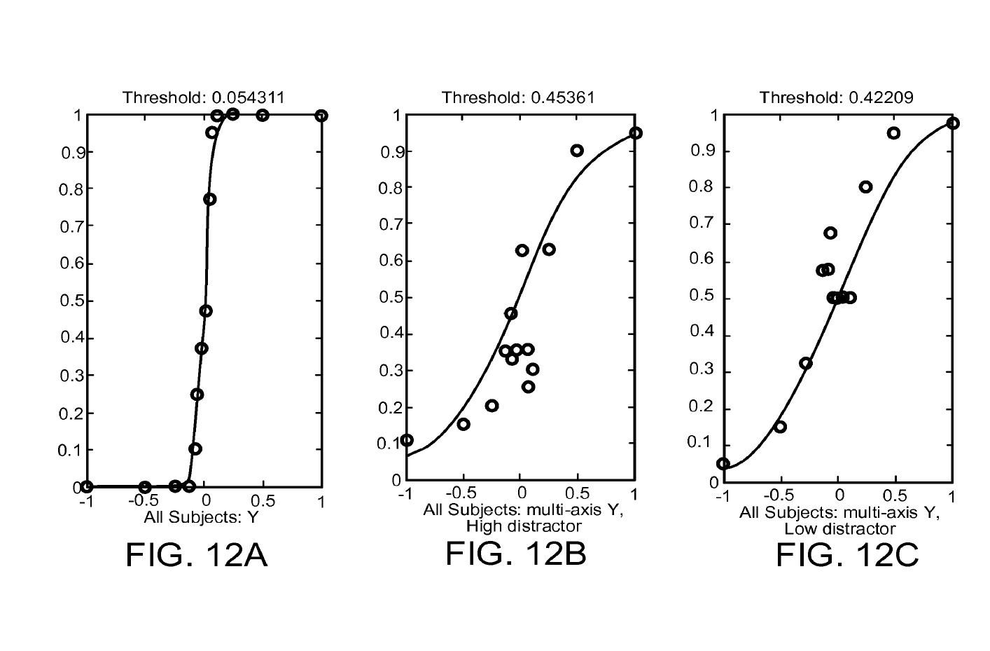

In another experiment, the effect of a distracting motion prior to providing a motion stimulus, was tested. In this case, four subjects were provided with a series of ten sequential single-cycle sinusoidal (5 Hz) acceleration motion stimuli—each 0.2 s in duration. One of these was translation to the left or right (y-axis direction recognition task) that varied in acceleration amplitude between −1 m/s/s to +1 m/s/s. Eight of the ten motion stimuli included two pitch tilts (0.1° each, which corresponds to a peak velocity of 2°/s and angular acceleration magnitude of 32°/s/s), two roll tilts (0.1° each), two yaw rotations (0.1° each), and two z-axis translations (0.6 mm each, which corresponded to a peak velocity of 6.4 mm/s and acceleration magnitude of 200 mm/s/s). The other motion was a forward/backward translation, which was either 0.6 mm (“low-amplitude”) or 1.2 mm (“high-amplitude”). The peak velocity of the x-axis motion always preceded the peak velocity of the y-axis motion by 0.2, 0.4, 0.6, 0.8, or 1.0 s. Translations along the x-axis or y-axis were never first or last. The results for the y-axis translation threshold are shown in FIGS. 12A-C. All rotations were about axes that intersected in the middle of the head at the level of the ears. Each of these motion stimuli was above the threshold measured when the stimuli were provided individually.

FIGS. 12A-C show average y-translation psychometric function across the four subjects for 3 different conditions. FIG. 12A is a plot obtained with no preceding distracting motion. The plot in FIG. 12B was obtained with a high-amplitude x-axis distracting motions, and the plot in FIG. 12C was obtained with low-amplitude x-axis distracting motions. Thresholds for y-translation with high-amplitude and low-amplitude distracting motions were indistinguishable and were both substantially greater than the threshold obtained with no preceding motion. In this example, the threshold was 0.05 m/s/s (0.32 m/s peak velocity) when the y-translation was presented in isolation. The thresholds were 0.45 m/s/s (2.87 cm/s) and 0.42 m/s/s (2.68 cm/s) when preceded by a high-amplitude or low-amplitude distracting motion, respectively. These results demonstrate that the y-translation threshold increased by almost an order of magnitude when immediately preceded by threshold-level motion in directions other than the y-axis translation threshold that was assayed.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-7. (canceled)

8. A method for estimating a vestibular function of a subject, the method comprising:
moving the subject along a first direction parallel to the direction of gravity;
receiving a first input set from the subject, the first input set indicating the subject's perception of the first direction;
estimating a first parameter related to a first vestibular function of the subject based on the first input;
changing an orientation of the subject with respect to the earth;
moving the subject along a second direction after changing the orientation of the subject;
receiving a second input set from the subject, the second input set indicating the subject's perception of the second direction;
estimating a second parameter related to a second vestibular function of the subject based on the second input; and
determining a relationship between the first parameter and the second parameter.

9. The method of claim 8, wherein the first direction and the second direction are substantially similar directions in a head coordinate of the subject.

10. The method of claim 8, wherein the second direction is different from the direction of gravity.

11. The method of claim 8, wherein the first direction and the second direction are substantially different directions in a head coordinate of the subject.

12. The method of claim 11, wherein the second direction is parallel to the direction of gravity.

13. The method claim 8, wherein determining the relationship includes comparing a magnitude of the first parameter and the second parameter.

14. The method of claim 8, further comprising:
evaluating whether the subject is a normal subject, a vestibular patient, or a malingerer based on the determined relationship between the first parameter and the second parameter.

15. The method of claim 8, further comprising:
producing a first vestibulogram based on the first parameter;
producing a second vestibulogram based on the second parameter; and
determining a relationship between the first vestibulogram and the second vestibulogram.

16. The method of claim 15, wherein determining the relationship between the first vestibulogram and the second vestibulogram includes calculating a correlation function of the first vestibulogram and the second vestibulogram.

17. The method of claim 15, further comprising:
evaluating whether the subject is a normal subject, a vestibular patient, or a malingerer based on a correlation between the first vestibulogram and the second vestibulogram.

18. A method for estimating a vestibular function of a subject, the method comprising:
moving the subject along a direction parallel to the direction of gravity;
receiving an input set from the subject, the input set indicating the subject's perception of the motion;
estimating a first parameter related to a vestibular function of the subject based on the received input;
determining a relationship between the estimated parameter and the predetermined parameter; and
evaluating whether the subject is a normal subject, a vestibular patient, or a malingerer based on the relationship.

19. The method of claim 18, wherein the subject is evaluated to be a vestibular patient when the estimated parameter is significantly greater than the predetermined parameter or a malingerer when the estimated parameter is not significantly greater than the predetermined parameter.

20. (canceled)

21. A method for estimating a vestibular function of a subject, the method comprising:
moving the subject along a direction; and
receiving an input from the subject, the input indicating the subject's perception of the direction;
wherein the input includes a binary response and a confidence rating, wherein the confidence rating is any of a quasi-continuous rating, a binary rating, a N-level discrete rating, or a wagering rating.

22. (canceled)

23. The method of claim 21, further comprising:
fitting the confidence rating to a distribution function; and
determining a next motion of the subject based on the fit.

24. The method of claim 21, further comprising:
fitting the confidence rating to a distribution function to improve the estimation of the vestibular function.

25. The method of claim 21, further comprising:
determining a correlation between the binary response and the confidence rating; and
evaluating the subject to be a malingerer if the determined correlation is different from a predetermined value.

26. A method for estimating a vestibular function of a subject, the method comprising:
moving the subject along a direction; and
receiving an input from the subject, the input indicating the subject's perception of the direction, wherein the input includes a binary response;
measuring vestibulo-ocular reflex (VOR) of the subject; and
producing VOR data from the measured VOR.

27. The method of claim 26, further comprising:
fitting the VOR data to a distribution function; and
determining a next motion of the subject based on the fit.

28. The method of claim 26, further comprising:
fitting the VOR data to a distribution function to improve the estimation of the vestibular function.

29. The method of claim 26, further comprising:
determining a correlation between the binary response and the VOR data; and
evaluating the subject to be a malingerer if the determined correlation is different from a predetermined value.

30. (canceled)
